(19) United States
(12) Patent Application Publication
(10) Pub. No.: US 2026/0193650 A1
(43) Pub. Date: Jul. 9, 2026

(54) Enhanced Mutants of a Bacterial Suppressor Tyrosyl tRNA and Uses Thereof

(71) Applicant: TRUSTEES OF BOSTON COLLEGE, Chestnut Hill, MA (US)

(72) Inventors: Abhishek Chatterjee, Lexington, MA (US); Delilah Jewel, Brighton, MA (US); Zeyi Huang, Brighton, MA (US); Rachel Huang, Brighton, MA (US)

(21) Appl. No.: 19/132,203

(22) PCT Filed: Nov. 27, 2023
(86) PCT No.: PCT/US2023/081104

§ 371 Date: May 22, 2025

Related U.S. Application Data
(60) Provisional application No. 63/385,109, filed on Nov. 28, 2022.

(51) Int. Cl. C12N 15/113; C12N 15/10; C12N 15/86; C12P 21/00

(52) U.S. Cl. CPC C12N 15/113; C12N 15/1058; C12N 15/86; C12P 21/00; C12N 2750/14143

(57) ABSTRACT

Compositions and methods are described demonstrating the ability to 1) use a virus assisted directed evolution platform to significantly improve the activity of engineered nonsense-suppressor tRNAs in mammalian cells, 2) provide mutants of E. coli tyrosyl-tRNAs that show remarkably improved Uaa incorporation efficiency in mammalian cells, and 3) use these tRNAs to express recombinant proteins in mammalian cells incorporating Uaas at significantly improved yields.

### RELATED APPLICATIONS

This application claims the benefit under 35 U.S.C. § 119(e) of U.S. Provisional Application No. 63/385,109, filed on Nov. 28, 2022, which is incorporated herein by reference in its entirety.

### GOVERNMENT SUPPORT

This invention was made with Government support under contract number R35 GM136437 awarded by the National Institutes of Health (NIH), and under contract number 1817893 awarded by the National Science Foundation (NSF). The Government has certain rights in the invention.

### INCORPORATION BY REFERENCE OF AN ELECTRONIC SEQUENCE LISTING

A Sequence Listing conforming to the rules of WIPO Standard ST.26 is hereby incorporated by reference. Said Sequence Listing has been filed as an electronic document encoded as XML in UTF-8 text. The electronic document, created on Oct. 21, 2023, is entitled “0342.0015WO1_ST26.xml”, and is 52,933 bytes in size.

### BACKGROUND OF THE INVENTION

Site-specific incorporation of unnatural amino acids (referred to herein as UAAs or Uaas) holds much potential to probe and engineer the biology of mammalian cells. Central to this technology is a nonsense-suppressing aminoacyl-tRNA synthetase (aaRS)/tRNA pair, which is engineered to charge the Uaa of interest without cross-reacting with any of its host counterparts. Such “orthogonal” aaRS/tRNA pairs are typically imported into the host cell from a different domain of life. The performance of the heterologous suppressor tRNA is often suboptimal in the new host, given it must directly interact with a nonnative translation system. Indeed, several studies have confirmed that Uaa incorporation efficiency in mammalian cells is limited by the poor performance of the heterologous suppressor tRNAs, which must be massively overexpressed for acceptable efficiency of Uaa incorporation. While such high levels of tRNA expression can be achieved through transient transfection in specific mammalian cell lines that exhibit high transfection efficiency, it is challenging to do so in difficult-to-transfect cells (e.g., primary cells, neurons, stem cells, etc.). Moreover, it makes generation of stable suppressor cell-lines (that express engineered aaRS/tRNA from the genome) very challenging, as hundreds of copies of tRNA gene must be inserted into the genome to reach sufficient nonsense suppression/Uaa incorporation efficiency.

The ability to overcome the suboptimal performance of the suppressor tRNA will significantly improve the robustness of the Uaa mutagenesis technology, facilitating advanced applications such as facile generation of stable suppressor cell lines capable of Uaa incorporation and simultaneous incorporation of Uaas at multiple sites in the same protein.

### SUMMARY OF THE INVENTION

This invention describes the directed evolution of a bacterial tyrosyl-tRNA (wild-type Tyr-tRNA as shown in SEQ ID NO:1), to generate mutants with significantly higher suppressor activity for unnatural amino acid (UAAs) (also referred to herein as a non-canonical amino acid (NCAA), or an amino acid analog) incorporation into proteins produced (e.g., expressed) in mammalian cells. UAA incorporation is achieved in response to nonsense (suppressor) codons (mainly TAG or TGA) using an engineered aminoacyl-tRNA synthetase (aaRS)-tRNA pair. This pair is imported, transfected or introduced, into the host cell from a different domain of life (i.e, orthogonal to the cell's native tRNA/aaRS pair) to ensure that it does not cross-react with its endogenous counterparts. However, due to its heterologous origin, the foreign pair does not efficiently interface with the translation machinery of the host, thus limiting the efficiency at which it incorporates UAAs. The problem of poor efficiency of the heterologous UAA-incorporating aaRS/tRNA pairs significantly limits the scope of the UAA-incorporation technology in mammalian cells. It reduces the yields of recombinant proteins incorporating UAAs, and precludes advanced applications such as site-specific incorporation of multiple UAAs into a protein.

It has been previously shown that the poor efficiency of the tRNA (and not the aaRS) limits the overall UAA incorporation efficiency in mammalian cells. (see Roy, G. et al. “Development of a high yielding expression platform for the introduction of non-natural amino acids in protein sequences”, mAbs 12, 1684749, doi:10.1080/19420862.2019.1684749 (2020); Schmied, W. H., Elsasser, S. J., Uttamapinant, C. & Chin, J. W. “Efficient multisite unnatural amino acid incorporation in mammalian cells via optimized pyrrolysyl tRNA synthetase/tRNA expression and engineered eRF1”, Journal of the American Chemical Society 136, 15577-15583 (2014); Zheng, Y., Lewis, T. L., Jr., Igo, P., Polleux, F. & Chatterjee, A. “Virus-Enabled Optimization and Delivery of the Genetic Machinery for Efficient Unnatural Amino Acid Mutagenesis in Mammalian Cells and Tissues, ACS Synthetic Biology, doi:10.1021/acssynbio.6b00092 (2016)).This is because the tRNA must directly interface with numerous translation components from the foreign host cell (e.g., elongation factors, ribosome), while the endogenous aaRS just needs to charge its cognate tRNA in the cell.

To overcome the poor efficiency of the heterologous nonsense suppressing tRNA, mammalian cell based directed evolution platform was developed (see WO2020/257668, the teachings of which are herein incorporated by reference, describes virus-assisted directed evolution of tRNAs (VADER) in mammalian cells, which employs a double-sieve selection scheme to facilitate single-step enrichment of active-yet-orthogonal tRNA mutants from naïve libraries). This platform couples the activity of the UAA incorporating aaRS/tRNA pair to the proliferation of adeno-associated virus (AAV), a small human virus. The portion of the suppressor tRNA that interfaces with the host translation system was randomized to create a large library of mutants. This naïve library is encoded in the AAV genome (each virus carries a unique tRNA mutant). This AAV-encoded tRNA mutant library is used to infect human cells at a low virus-to-cell ratio), such that each cell receives no more than one virus (e.g., one virion per cell). The cells are also supplied in trans with all the other genetic machinery/elements that the AAV needs to produce progeny virus. However, the gene encoding AAV capsid is inactivated with a TAG nonsense codon. Thus, only if the incoming/introduced virus encodes a highly active TAG-suppressing tRNA is the AAV virus able to produce full-length capsid protein and produce progeny virus. Those encoding inactive tRNAs are unable to replicate, as these fail to produce full-length capsid protein from TAG-inactivated capsid gene. Consequently, sequencing the genome of the progeny virus allows the identification all the tRNA mutants that are active (e.g, can actively suppress the TAG codon).

As described herein, this technology is used to generate acceptor stem mutant libraries of the E. coli Tyr-tRNA. Subjecting this library to the aforementioned selection scheme enables the identification of suitable mutant Tyr-tRNAs that show significantly higher suppressor activity in mammalian cells. These tRNAs provide much higher yield of UAA incorporation (e.g., tyrosine and phenylalanine analogs) in mammalian cells and facilitate incorporation of multiple UAAs into the same protein. Also as described herein, the higher activity of the most efficient mutant Tyr-tRNA is specific for mammalian cells, alluding to an improved interaction with the unique mammalian translation apparatus.

The mutant Tyr-tRNAs (also referred to herein as tyrosyl-tRNA variants) described herein exhibit increased suppressor activity over other suppressor tRNAs derived through standard methods alone, and therein perform significantly more efficiently (e.g., with higher yield) for UAA incorporation in mammalian cells. The improved efficiency of these tRNAs provide significant advantage over the current form of the technology, which is limited in the levels of expression yield that can be obtained for UAA-incorporated proteins expressed in mammalian cells (e.g., full-length antibodies).

The improved mutants of Tyr-tRNA (mutants with increased suppressing activity over the counter-part wild-type tRNA activity) can be used, for example, for improving the yields of UAA-incorporated protein in mammalian cells (e.g., antibodies and other therapeutically related proteins) and creating improved viral vectors that deliver the genetic machinery for UAA incorporation into mammalian cells and tissues.

As the tyrosyl-tRNA variants described herein are more efficient in their suppressing activities, fewer copies are required per genome. Currently, including multiple tRNA copies (to achieve high enough expression of tRNAs) often leads to genome instability of viral vectors or stable cell lines. Creating stable cell lines for protein expression incorporating UAAs is essential for production in commercial scale, but the requirement of encoding a large number of tRNA copies per genome makes it challenging to generate such cell lines, where the UAA-incorporation machinery is stably integrated in the mammalian genome.

The present invention encompasses tyrosyl-tRNA molecules/compositions wherein the tRNA anti-codon loop is modified (e.g., mutated) to specifically bind to (e.g., recognize) an amber (UAG/TAG) codon as described herein. tRNA compositions comprising the ochre codon (TAA/UAA) or the opal codon (UGA/TGA) are also encompassed by the present invention. In particular, the present invention encompasses compositions wherein the variant tRNA is the E. coli tyrosyl tRNA, or another homologous bacteria-derived tRNA.

Encompassed by the present invention is a composition comprising a variant bacterial tyrosyl-suppressor tRNA, wherein the variant tRNA has increased suppressor activity to incorporate an unnatural amino acid (amino acid analog) into a mammalian protein expressed in mammalian cells relative to its wild-type/native/endogenous cellular counterpart tRNA and a viral vector comprising the variant bacterial tyrosyl-suppressor tRNA. The biological activity (e.g., the suppressor activity of the variant tRNA is increased over the wild type tRNA by about 2.5 to about 100 fold, or about 5 to about 50-fold, or about 5 to about 25-fold, or more specifically about 10-15-fold, for example about 12-fold. In a preferred embodiment, the variant bacterial Tyr-tRNA is derived from an E. coli tRNA, wherein the tRNA is a tyrosyl-tRNA (tRNA^{Tyr}) comprising SEQ ID NO:1, or a Tyr-tRNA molecule comprising at last about 80, 85, 90, 95, 96, 97, 98 or 99% sequence identity to the Tyr-tRNA sequence SEQ ID NO:1. Specifically encompassed herein are the variant tRNA^{Tyr} comprising nuclei acid sequences as shown in FIG. 7 (SEQ ID NOS:3-24) and FIG. 9 (SEQ ID NOS: 25-31. The variant Tyr-tRNAs or viral vectors comprising the variant Tyr-tRNA compositions described herein are suitable to incorporate any suitable tyrosine or phenylalanine analog into a peptide or protein. Tyrosine and phenylalanine are aromatic amino acids with structural similarity as both possess aromatic, hydrophobic side chains (tyrosine having a 4-hydroxybenzyl group at the side chain and phenylalanine having a benzyl side chain). (See for example, Ledley, F. D. et al., “Homology Between Phenylalanine and Tyrosine Hydroxylases Reveals Common Structural and Functional Domains”, Biochem. Vol. 24:14 (1985)). More specifically, the closely related structures differ by a —OH group, with a hydroxyl group on tyrosine but not phenylalanine. Thus, it is reasonable to believe that the tyrosyl-tRNA variants described herein would function to site- specifically incorporate phenylalanine analogs into a protein as well as tyrosine analogs. Examples of tyrosine analogs suitable for incorporation are shown as structures 1-4 in FIG. 3A and structures 5-11 in FIG. 11A. Other tyrosine and phenylalanine analogs are known to those of skill in the art.

Also encompassed herein is a cell comprising the variant Tyr-tRNA or the viral vector stably integrated into the genome of the cell as described herein, wherein the cell is a mammalian cell.

More specifically, the viral vector comprising the variant Tyr-tRNA is adeno-associated virus (AAV) comprising an essential viral protein (i.e., a proteins required for viral replication. In one embodiment, the essential viral protein is the capsid protein (CAP) comprising SEQ ID NO:2 of the adeno associated virus, wherein the CAP protein is mutated to include a stop codon at position 454 of the protein. The composition or viral vector can comprise a variant tRNA wherein the variant is a tyrosyl-tRNA (tRNA^{Tyr}) comprising a sequence selected from the group consisting of: SEQ ID NOS: 3 through 31, or a nucleic acid sequence with at least about 90% sequence identity to any of the full-length sequences of SEQ ID NOS: 3-31. The composition or viral vector of the present invention can further comprise a tyrosine amino acid analog, for example, OmeY or any of the structures 1-11 as described herein.

Also encompassed by the present invention is a cell comprising the viral vector described herein, specifically wherein the vector is stably integrated into the genome of the cell. In a specific embodiment of the present invention the cell is a mammalian cell. The cell comprises the viral vector adeno-associated virus (AAV) and the essential viral protein is a TAG-mutant of Cap (SEQ ID NO:2).

Further, the mammalian cell can comprise plasmids or genetic elements encoding or providing the following elements required for viral replication: a) a protein essential for viral replication, wherein a nonsense codon is inserted into a protein sequence rendering viral replication dependent on the activity of the variant suppressor tRNA; b) a cognate Uaa RNA Synthetase (UaaRS); and c) genetic components required for viral replication. More specifically, the mammalian cell comprises the cognate aaRS is E.coli^{tyr}RS.

Also encompassed herein is a method of virus-assisted directed evolution (also referred to herein as VADER) of orthogonal suppressor Tyr-tRNA variants of interest with increased suppressor activity relative to the wild-type suppressor tRNA, wherein replication of the virus in mammalian cells requires expression of an essential protein dependent on the activity of the tRNA variant of interest. In one embodiment the method comprises the steps of:

A method of virus-assisted directed evolution of orthogonal suppressor tRNA variants of interest with increased biological activity relative to the wild-type suppressor tRNA, the method comprising the steps of:
a) encoding a library of suppressor tRNA variants of interest in a virus genome;
b) infecting a population of mammalian host cells with the virus vectors at low multiplicity of infection (MOI) and maintaining the population of cells under conditions suitable for virus replication in the cells, wherein virus replication in mammalian cells requires expression of an essential protein dependent on the activity of the tRNA variant of interest; and
c) harvesting and selectively amplifying the virus progeny encoding active tRNA variants to remove cross-reactive tRNA molecules, whereby orthogonal suppressor tRNA variants with increased biological activity are recovered.

More specifically, encompassed herein is a method of virus-assisted directed evolution of orthogonal nonsense suppressor tRNA variants of interest with increased biological activity relative to the wild-type suppressor tRNA activity, wherein the replication of the virus in mammalian cells requires expression of a nonsense mutant of an essential viral protein which is dependent on the activity of the suppressor tRNA variant of interest, the method comprising the step of:
a) encoding a library of suppressor Tyr-tRNA variants in a virus genome;
b) infecting a population of mammalian host cells with the virus vectors at low multiplicity of infection (MOI);
c) subsequently transfecting the population of mammalian host cells with additional plasmids, or genetic elements, wherein the plasmids or genetic elements comprise:
i) a protein essential for viral replication, wherein a nonsense codon is inserted into the protein sequence rendering viral replication dependent on the activity of the variant suppressor tRNA;
ii) a cognate Uaa RNA Synthetase (UaaRS); and
iii) genetic components required for viral replication;
d) substantially simultaneously adding a suitable unnatural amino acid analog such as a tyrosine or phenylalanine analog to the culture media;
e) maintaining the infected/transfected cells in the media under conditions suitable for replication of the virus;
f) harvesting the cells and isolating virus progeny; and
g) lysing the recovered virus and amplifying tRNA variants contained in the lysate, whereby orthogonal suppressor Tyr-tRNA variants with increased biological suppressor activity are recovered.

After amplification, the variant tRNA molecules can be sequenced to determine the nucleic acid sequences of the variants.

Optionally, the method can comprise the additional steps of: labeling the virus isolated in step f) with a purification handle attached through a photocleavable linker; and recovering labeled virus through enrichment followed by release using irradiation at a suitable wavelength prior to the final method step.

Optionally, the methods described herein can also comprise sequencing the suppressor tRNA variants of the library produced as described herein before encoding or introducing into the virus.

The host cells of the described methods can further comprise plasmids encoding a protein essential for viral replication, wherein a nonsense codon is inserted into a protein sequence rendering viral replication dependent on the activity of the variant suppressor tRNA; b) a cognate Uaa RNA Synthetase (UaaRS); and c) genetic components required for viral replication. It is noted that although plasmids are exemplified other genetic elements can be used to provide the components required for viral replication such as DNA delivery via additional viruses. Such methods are known to those of skill in the art.

Specifically encompassed herein are methods of producing a protein or a peptide of interest expressed in a mammalian cell with one, or more, tyrosyl or phenylalanine analogs at specified/designated positions (site-specifically incorporated) into the protein, the method comprising culturing the cell in a culture medium under conditions suitable for growth, wherein the cell comprises a nucleic acid that encodes a protein with one, or more, amber selector codons, and wherein the cell further comprises an Ec-tRNA^{Tyr} that recognizes the amber selector codon(s), and contacting the cell culture medium with one, or more, tyrosyl analogs under conditions suitable for incorporation of the one, or more, tyrosyl or phenylalanine analogs into the protein in response to the selector codon, thereby producing the protein with one, or more tyrosyl or phenylalanine analogs.

More specifically, the method of site-specifically incorporating one, or more, tyrosine or phenylalanine analogs into a protein or peptide of interest comprises the following steps:
a. culturing the mammalian cell in a culture medium under conditions suitable for growth, wherein the cell comprises a nucleic acid that encodes a protein or peptide of interest with one, or more, amber, ochre or opal selector codons, wherein the cell comprises a nucleic acid that encodes a protein or peptide of interest with one, or more, amber, ochre or opal selector codons at specific sites in the protein or peptide,
wherein the cell also comprises a variant E. coli-derived tRNA^{Tyr} with increased biological activity that recognizes the selector codon, and the cell further comprises a suitable cognate E. coli Tyr-RNA Synthetase; and
b. contacting the cell culture medium with one, or more, tyrosyl or phenylalanine amino acid analogs under conditions suitable for incorporation of the one, or more, tyrosine analogs into the protein or peptide of interest in response to the selector codon,
thereby producing the protein or peptide of interest with one, or more tyrosine or phenylalanine analogs located at specific pre-determined sites within the protein.

The variant Ec-tRNA^{Tyr} molecules described herein comprise any one of the following nucleotide sequences as shown in FIG. 7 or 9 (SEQ ID NOS:3-31), or a nucleic acid sequence with at least 90% sequence identity over the full-length sequences of SEQ ID NOS; 3-31.

Also encompassed herein is a kit for producing a protein or peptide of interest in a mammalian cell comprising one, or more tyrosine analogs incorporated into the peptide or proteins, wherein the protein or peptide comprises one, or more tyrosine or phenylalanine analogs, the kit comprising: a container containing a polynucleotide sequence encoding variant E. coli derived tRNA^{Tyr} with increased biological activity that recognizes a selector codon in a nucleic acid of interest in a cell; and a container containing a polynucleotide sequence encoding E. coli Tyr-tRNA synthetase. The variant Ec-tRNA^{Tyr} molecules described herein comprise any one of the following nucleic acid sequences as shown in FIG. 7 or 9 (SEQ ID NOS:3-31). The kit can also include instructions for producing the protein or peptide of interest.

As described herein, the present invention also encompasses a genetically-engineered mammalian cell with a stably integrated variant suppressor tRNA^{Tyr} for Uaa incorporation. Specifically, the genetically-engineered mammalian cell comprises less than 250, 200, 150, 100, 75, 50 copies of a gene encoding a variant suppressor tRNA^{Tyr} capable of incorporating an unnatural amino acid into a protein of interest.

Further, the genetically-engineered mammalian cell comprises 25-250, 25-200, 25-150, 25-100, 25-75, 25-50, 50-250, 50-200, 50-150, 50-100, 50-75, 75-250, 75-200, 75-250, 75-100, 100-250, 100-200, or 100-150 copies of the gene encoding the suppressor tRNA. The variant suppressor tRNA^{tyr} of the cell comprises a nucleic acid sequence selected from the group consisting of: SEQ ID NOS: 3-31, or a sequence with at least about 90% sequence identity to any of the full-length sequences of SEQ ID NOS: 3-31.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1A-C show the general VADER selection scheme to obtain Try-tRNA mutants with increased suppressor activity. (A) Mammalian cells are infected with AAV2 encoding the tRNA library at low MOI. Plasmids encoding TAG-mutant of Cap, other genetic components needed for AAV replication, and the cognate aaRS are provided in trans by transfection in the presence of a suitable azido-Uaa. Active and orthogonal tRNA mutants facilitate generation of packaged progeny AAV2 incorporating the Uaa into their capsid, which are isolated by chemoselective biotin conjugation followed by streptavidin pulldown. (B) Two AAV2 vectors, encoding i) E. coli tRNATyr and mCherry (Tyr-mCherry), and ii) tRNAPyl and EGFP (Pyl-EGFP), were mixed in a 104:1 ratio and subjected to the VADER selection scheme using EcTyrRS and its substrate pAAF. (C) FACS analysis of the surviving population shows a >30,000-fold cumulative enrichment of Tyr-mCherry. Data shown as mean±s.d. (n=3 independent experiments).

FIG. 2A-D show a specific VADER scheme directed to the evolution of tRNATyr. (A) The randomization scheme to create the E. coli tRNATyr mutant library. (B) The consensus corresponding to 172 homologous bacterial tRNATyr sequences, which was used to guide the tRNATyr randomization scheme. (C) Consensus sequence of the tRNATyr mutants exhibiting the highest average enrichment (top 1%) following the VADER scheme (FIG. 1). (D) Efficiency of TAG suppression for the most active tRNATyr mutants using the EGFP-39TAG. The tRNA encoded in the pAAV plasmid (also harboring a wild-type mCherry reporter) was co-transfected into HEK293T cells with OMeY-selective EcTyrRS and EGFP-39TAG in the presence or absence of 1 mM OMeY. Expression of EGFP-39TAG was measured in cell-free extract, normalized relative to wild-type mCherry expression and plotted as a percentage of the normalized activity of wild-type tRNATyr. Data shown as mean±s.d. (n=3 independent experiments).

FIG. 3A-B show enhanced suppressor activity of two enhanced tRNA mutants for incorporating additional tyrosine analogs. (A) Structures of the tyrosine analogs 1-4 tested as described herein. (B) Relative incorporation efficiencies of these UAAs using two engineered tRNA(Tyr) as well as the corresponding wild-type.

FIG. 4A-F show a specific VADER scheme directed to the evolution of tRNA^{Tyr}. (A) A modified VADER scheme lacking the bioorthogonal capture step. Instead, the selective amplification is performed either in the presence or the absence of the cognate synthetase. Active and orthogonal tRNA mutants are identified by their selective enrichment in the presence of the cognate aaRS. (B) The randomization scheme to create the E. coli tRNA^{Tyr} mutant library. (C) The consensus corresponding to 172 homologous bacterial tRNA^{Tyr} sequences, which was used to guide the tRNA^{Tyr} randomization scheme. (D) Sequence of the tRNA^{Tyr} mutants exhibiting the highest average enrichment following the VADER scheme shown in panel A in the presence of EcTyrRS. (E) Observed enrichment of each mutant in the tRNA^{Tyr} library upon subjecting them to the VADER selection scheme either in the presence of a cognate EcTyrRS mutant that charge OMeY, or MbPylRS (does not recognize tRNA^{TY}r). Each selection was performed in duplicate, and the normalized enrichment factors observed from each were plotted against each other. Nine out of the ten most enriched sequences in the presence of EcTyrRS did not show strong enrichment when MbPylRS was used instead (shown in green; sequences shown in panel D; the cross-reactive mutant is shown in red. (F) Efficiency of TAG suppression for these nine tRNA^{Tyr} mutants using the EGFP-39TAG. The tRNA encoded in the pAAV plasmid (also harboring a wild-type mCherry reporter) was co-transfected into HEK293T cells with OMeY-selective EcTyrRS and EGFP-39TAG in the presence or absence of 1 mM OMeY. Expression of EGFP-39TAG was measured in cell-free extract, normalized relative to wild-type mCherry expression and plotted as a percentage of the normalized activity of wild-type tRNA^{Tyr} Data shown as mean±s.d. (n=3 independent experiments).

FIG. 5 shows a nucleotide sequence of wild-type E. coli tyrosyl tRNA (SEQ ID NO:1).

FIG. 6 shows an amino acid sequence of AAV2 Cap protein (VP1) (SEQ ID NO:2).

FIG. 7 shows the nucleic acid sequences of novel tyrosine tRNA mutants generated as described herein (SEQ ID NOS:3-24).

FIG. 8 A-C A library of tyrosyl tRNA was created for each nucleotide and was individually randomized to all possible combinations of SEQ ID NO:5 as shown in FIG. 8A. Additionally, the library also harbored mutants where each base pair within the tRNA were individually randomized to all possible combinations, maintaining base-pairing. This collection of mutants was subjected to virus-assisted selection scheme described herein, that enriches mutants that are active but not cross-reactive. Next-generation sequence analysis was used to measure average enrichment of each mutant derived from SEQ ID NO:5 (an indirect measure of their activity) (FIG. 8B). These were plotted using WebLogo (Crooks, G. E. et al., Genome. Res. 14:1188-1190 (2004) to reveal how each mutation at each position either positively or negatively impacts tRNA activity (FIG. 8C). These results highlight important elements of the tRNA that cannot be mutated, but also reveal potential alterations that may result in higher activity.

FIG. 9 shows the sequences of variant tyrosyl-tRNA molecules (e.g., mutant tyrosyl-tRNA (SEQ ID NOS: 25-31) that show enhanced activity as identified herein. Bolded fonts are used to highlight the mutations/sequence alterations relative to the WT sequence.

FIG. 10 shows the activity of the mutant sequences SEQ ID NOS: 25-31 measured using an EGFP-39-TAG reporter. The Y-axis shows fold improvement relative to the wild-type tRNA-Tyr.

FIG. 11A-B show enhanced suppressor activity of two enhanced tRNA mutants for incorporating additional tyrosine analogs. (A) Relative incorporation efficiencies of these UAAs using two engineered tRNA(Tyr) as well as the corresponding wild-type. (B) Structures of the tyrosine analogs 5-11 tested as described herein.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Site-specific incorporation of unnatural amino acids (Uaas) into proteins in mammalian cells holds much potential to enable both basic science as well as biotechnology applications. (See Chin, J. W. Expanding and reprogramming the genetic code. Nature 550, 53-60, doi:10.1038/nature24031 (2017); Italia, J. S. et al. Expanding the genetic code of mammalian cells. Biochemical Society transactions 45, 555-562, doi:10.1042/bst20160336 (2017); Young, D. D. & Schultz, P. G. Playing with the Molecules of Life. ACS Chem Biol 13, 854-870, doi:10.1021/acschembio.7b00974 (2018); Manandhar, M., Chun, E. & Romesberg, F. E. Genetic Code Expansion: Inception, Development, Commercialization. J Am Chem Soc 143, 4859-4878, doi:10.1021/jacs.0c11938 (2021).

Central to this technology is a nonsense-suppressing aminoacyl-tRNA synthetase (aaRS)/tRNA pair which is engineered to charge the Uaa of interest without cross-reacting with any of its host counterparts. The biology of tRNAs is complex and multifaceted involving expression, processing, post-transcriptional modifications, cellular stability, interaction with the cognate aaRS and the components of the translational system (e.g., elongation factors and the ribosome), etc. (Soll, D. & RajBhandary, U. L. tRNA: Structure, Biosynthesis, and Function. (ASM Press, 1995).

How these different facets of tRNA biology contribute to the poor performance of foreign suppressor tRNAs is poorly understood, which makes it challenging to develop better variants through rational design. Although a semi-rational approach was recently used with some success to improve the activity of pyrrolysyl-tRNA in mammalian cells, (Serfling, R. et al. Designer tRNAs for efficient incorporation of non-canonical amino acids by the pyrrolysine system in mammalian cells. Nucleic acids research 46, 1-10, doi:10.1093/nar/gkx1156 (2018), it is challenging to generalize such strategies for tRNA engineering. Directed evolution has been used with much success to develop improved orthogonal suppressor tRNA mutants in E. coli. Chatterjee, A., Xiao, H. & Schultz, P. G. Evolution of multiple, mutually orthogonal prolyl-tRNA synthetase/tRNA pairs for unnatural amino acid mutagenesis in Escherichia coli. (Proceedings of the National Academy of Sciences of the United States of America 109, 14841-14846, doi:10.1073/pnas.1212454109 (2012); Chatterjee, A., Xiao, H., Yang, P. Y., Soundararajan, G. & Schultz, P. G. A tryptophanyl-tRNA synthetase/tRNA pair for unnatural amino acid mutagenesis in E. coli. Angewandte Chemie (International ed. in English) 52, 5106-5109, doi:10.1002/anie.201301094 (2013).

This was made possible by the development of selection systems, which enables the enrichment of active and orthogonal suppressor tRNA mutants from large synthetic libraries. The ability to perform analogous tRNA evolution in mammalian cells has the potential to yield improved suppression systems, but no suitable platform is currently available. It is important to perform such directed evolution experiments in mammalian cells, instead of in lower organisms, where directed evolution is better established, to ensure that the tRNA mutants are selected based on their improved interactions with the unique mammalian translation system.

Existing directed evolution strategies in mammalian cells largely rely on stable integration of the target gene in a cell line, followed by the creation of sequence diversity through untargeted or targeted random mutagenesis. This approach is unsuitable for tRNA evolution for two reasons: A) To ensure a clear genotype-phenotype connection, it is essential to have no more than a single genomically integrated mutant tRNA gene per cell. However, it has been shown that a large number of tRNA genes (>100) are needed per cell to achieve detectable Uaa incorporation efficiency; B) The low mutagenic frequency associated with these strategies is poorly suited for tRNA evolution, given its small size (<100 bp). One particular challenge arises when diversifying the stem regions of a tRNA, which are most frequently targeted for directed evolution: any mutation must be accompanied by a matching mutation on the other side to retain base-pairing, the loss of which compromises tRNA secondary structure and activity. Capturing such rich sequence diversity within the small tRNA gene is practically only feasible using synthetic site-saturation mutagenesis libraries. To enrich suppressor tRNA variants that are orthogonal and active in mammalian cells from such synthetic libraries, we need the following: i) controlled delivery of the library members into mammalian cells, such that each cell receives a single variant; ii) a selection scheme that enriches the active tRNA mutants, and removes cross-reactive ones; and iii) the ability to identify the surviving mutants.

The feasibility of producing adeno-associated virus (AAV2) in mammalian cells, site-specifically incorporating Uaas into its capsid through amber suppression was recently reported. In particular, see the mammalian cell based directed evolution platform as described in WO2020/257668, the teachings of which are herein incorporated in their entirety by reference.

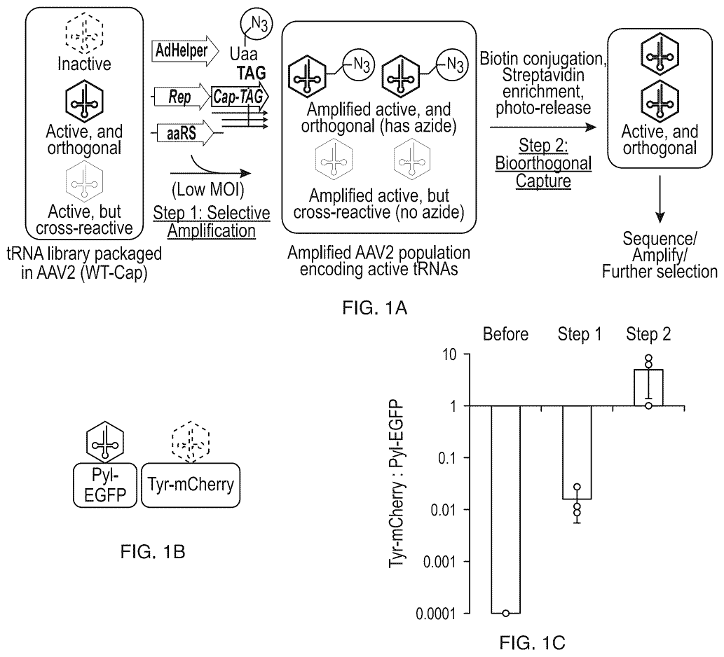

In this system, successful AAV2 production is dependent on the activity of the suppressor aaRS/tRNA pair, providing an attractive platform for virus-assisted directed evolution of tRNA (VADER) in mammalian cells. AAV2 also offers additional advantages such as the lack of known pathogenicity, and a small genome that is amenable to facile manipulations. An optimized double-sieve selection scheme (FIG. 1) for VADER that facilitates efficient one-step enrichment of active and orthogonal tRNA variants from naïve synthetic libraries was developed. Using VADER, mutants of M. mazei pyrrolysyl tRNA, which show significantly improved activity at lower expression levels were developed. Furthermore, coupling VADER with next-generation sequencing (NGS) analysis provided a global view of the evolutionary landscape, where performance of each tRNA mutant in the entire library can be simultaneously tracked.

Described herein are compositions comprising variant/mutant nonsense suppressing tRNA molecules (also referred to herein as suppressor tRNAs) having increased biological activity relative to the corresponding wild type suppressor tRNA molecule to incorporate an unnatural amino acid (Uaa or UAA) into a mammalian protein; expression vectors (e.g., viral vectors) encoding these variant tRNAs where the vectors are suitable for infecting mammalian cells; mammalian cells comprising these expression vectors (e.g., viral vectors); methods of producing suppressor tRNAs with increased biological activity using the virus-assisted directed evolution methods described herein; methods of using these tRNAs with increased activity to produce proteins with site-specifically incorporated unnatural amino acids and kits containing reagents comprising the variant tRNAs and other reagents required for producing such proteins.

In particular, the compositions of the present invention comprise, for example, a variant archaeal or bacterial nonsense suppressing tRNA molecule, wherein the orthogonal, active variant tRNA has increased activity to incorporate various unnatural amino acids (e.g., amino acid analogs) into a mammalian protein relative to its “wild type” counterpart suppressor tRNA. The term “wild type” counterpart tRNA as used herein means a suppressor tRNA molecule that has not been subjected to the virus-assisted directed evolution methods described herein to produce (select and enrich) a population of suppressor tRNA molecules having increased biological activity to incorporate a Uaa into a protein of interest in a site specific manner.

The activity of the variant tRNAs encompassed by the present invention is increased over the wild type tRNA, for example, by about 2.5 to about 100 fold, about 2.5 to about 80 fold, about 2.5 to about 50 fold about 2.5 to about 25 fold, about 2.5 to about 20 fold, about 2.5 to about 15 fold, about 2.5 to about 12 fold, about 2.5 to about 10 fold, about 2.5 to about 5 fold, about 5 to about 100 fold, about 5 to about 80 fold, about 5 to about 50 fold, about 5 to about 25 fold, about 5 to about 20 fold, about 5 to about 15 fold, about 5 to about 12 fold, about 5 to about 10 fold, about 10 to about 100 fold, about 10 to about 80 fold, about 10 to about 50 fold, about 10 to about 25 fold, about 10 to about 20 fold, about 10 to about 15 fold, about 10 to about 12 fold.

More specifically, described herein is the specific modification of VADER to develop improved mutants of the E. coli tyrosyl-tRNA, using a ‘single-sieve’ variant of NGS-coupled VADER. Improved mutants of E. coli tRNA_{CUA}^{Tyr} were developed by subjecting an acceptor-stem library to a modified VADER scheme lacking the bioorthogonal capture step. Importantly, this modification of VADER lacks the negative selection step.

These results suggest that it should be possible to extend the application of VADER to other suppressor tRNAs commonly used for genetic code expansion in mammalian cells. Additionally, it should be possible to adapt this platform for the directed evolution of other biological machinery for Uaa mutagenesis and beyond.

The references listed in this application are herein incorporated by reference in their entirety.

Without further elaboration, it is believed that one skilled in the art can, based on the above description, utilize the present invention to its fullest extent. The following specific embodiments and examples are, therefore, to be construed as merely illustrative, and not limitative of the remainder of the disclosure in any way whatsoever.

### Example 1: Directed Evolution of tRNA^{Tyr} Using a Single-Step Scheme

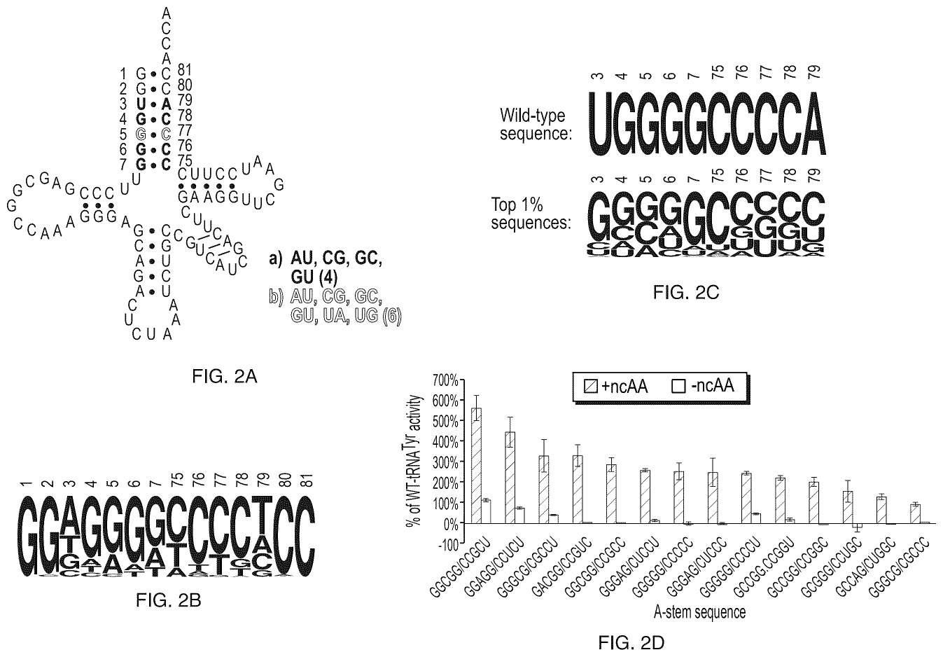

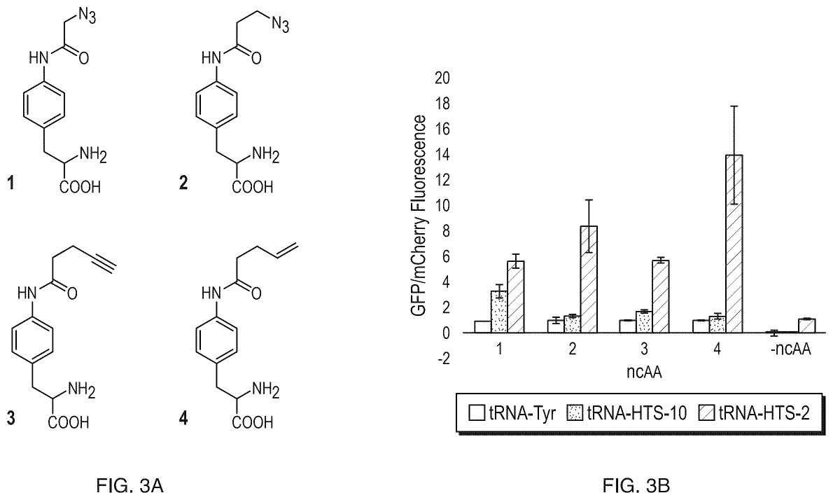

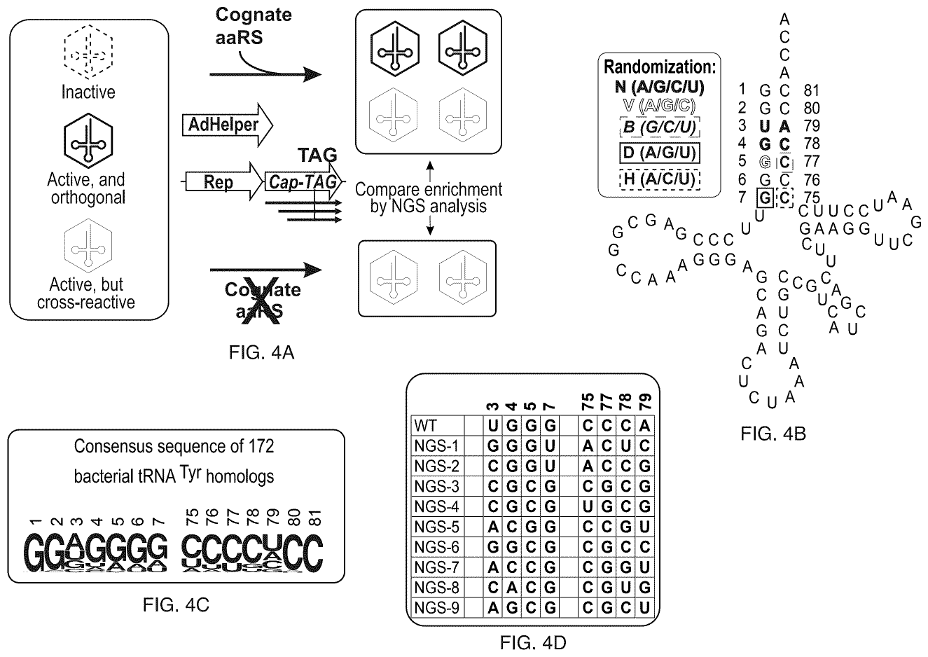

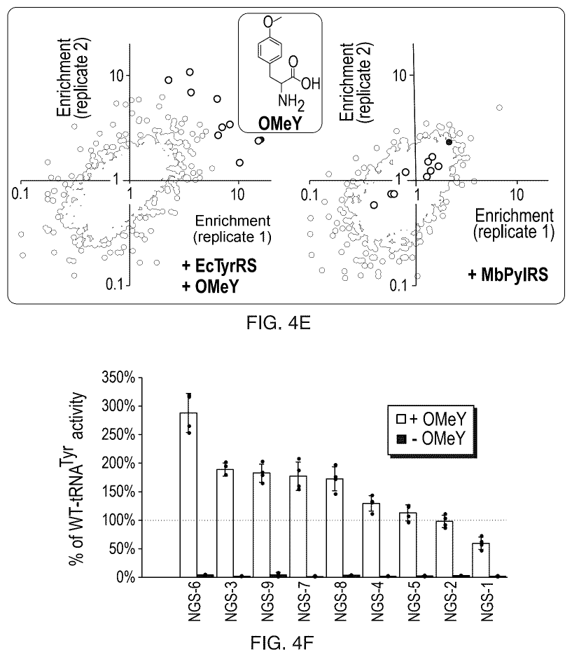

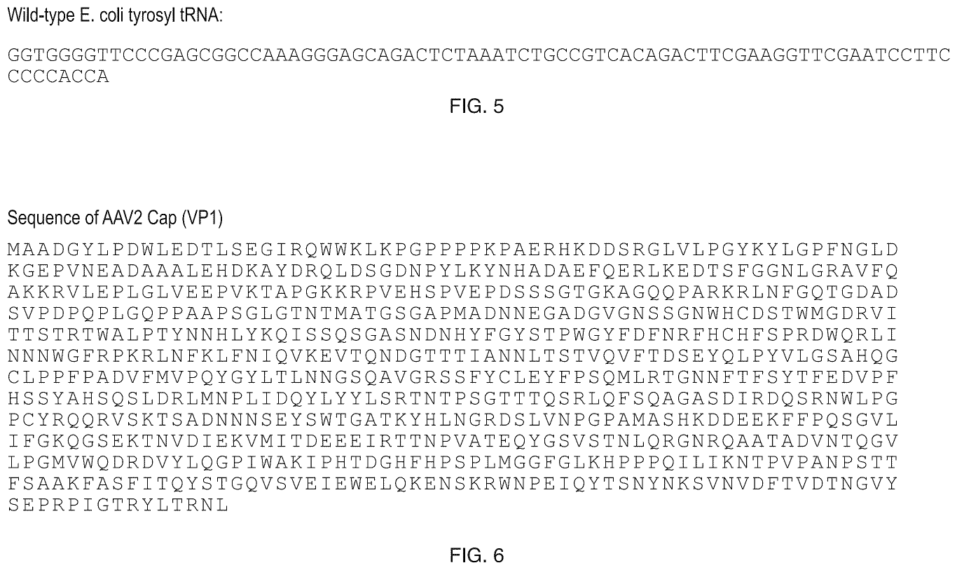

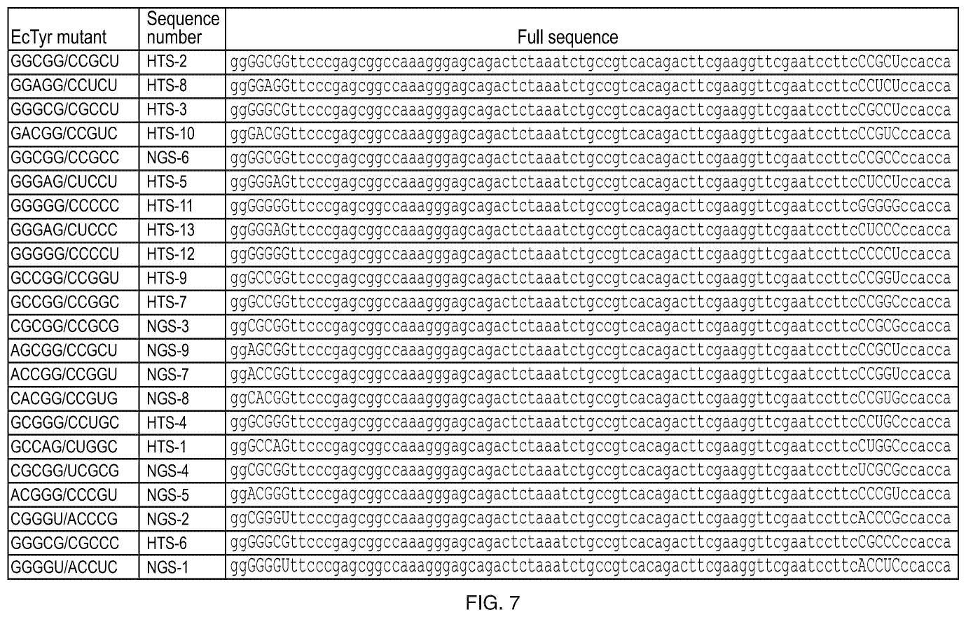

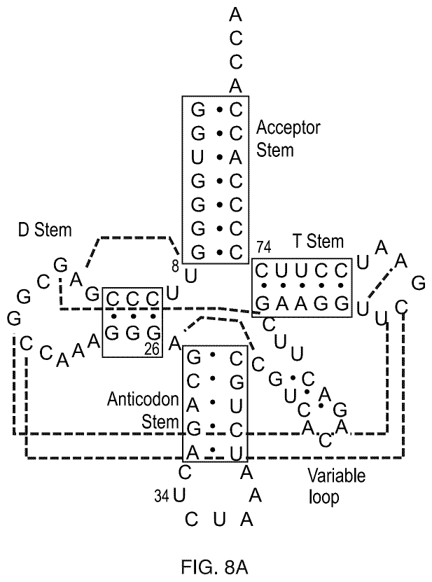

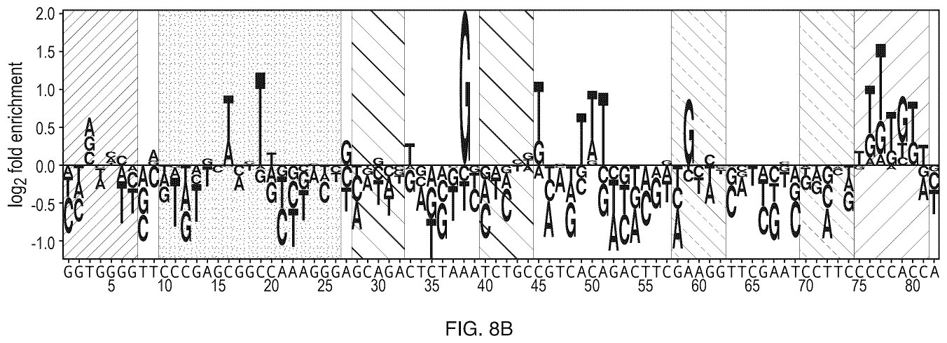

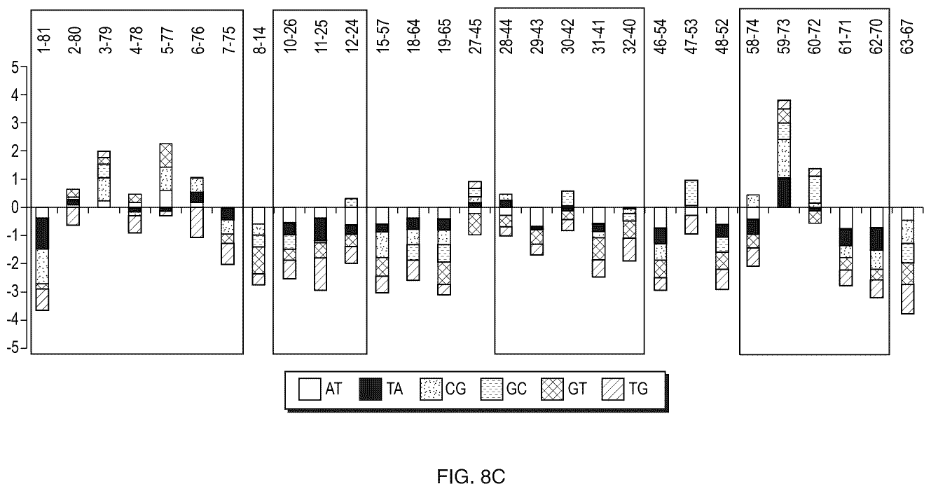

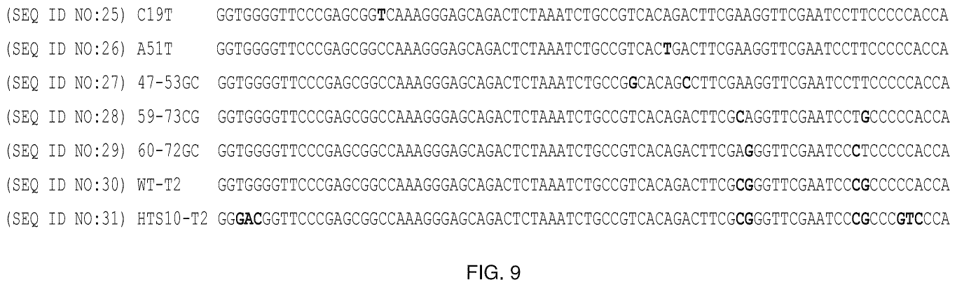

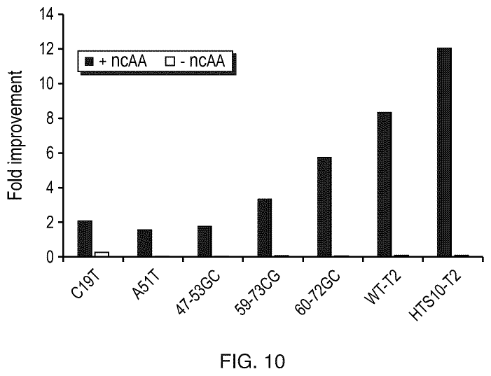

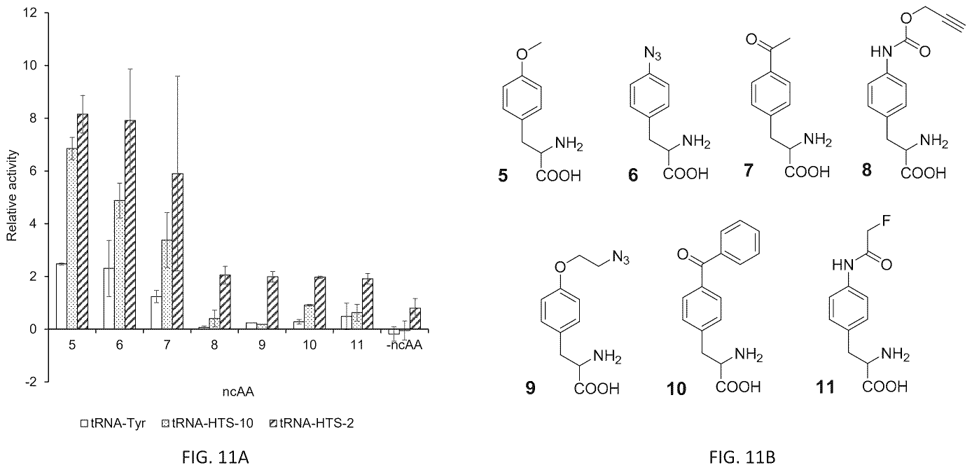

As described herein, the VADER scheme for the directed evolution was applied to the directed evolution of E. coli tyrosyl tRNA. It was first confirmed that the tRNA^{Tyr} is compatible with the VADER selection system. When a defined mixture of two AAV2 vectors encoding either tRNA^{Tyr} or tRNA^{Pyl} was subjected to the selective amplification step of VADER, in the presence of an established polyspecific EcTyrRS mutant that charges O-methyltyrosine (OMeY), the tRNA^{Tyr}-encoding virus was significantly enriched in the output. However, the use of the azide-bearing Uaa p-azidophenylalanine (pAzF) instead of OMeY, which can also be charged with the same polyspecific EcTyrRS, failed to generate packaged virus, possibly due to perturbations to the virus capsid. Although the absence of an azide-Uaa precludes the 2^{nd}-step of VADER involving bioorthogonal capture, which is designed to remove the cross-reactive tRNA population, an alternative scheme was devised to achieve the same goal (FIG. 2a). It was reasoned that active yet orthogonal tRNA mutants can be identified by their conditional enrichment upon performing the selective amplification step of VADER in the presence of the cognate synthetase, but not in its absence.

A library of approximately 20,000 mutants was generated by randomizing different bases in the acceptor stem (FIG. 2b), guided by the consensus sequence from 172 different bacterial tRNA^{Tyr} homologs (Jühling, F. et al. tRNAdb 2009: compilation of tRNA sequences and tRNA genes. Nucleic acids research 37, D159-162, doi:10.1093/nar/gkn772 (2009) (FIG. 2c) to retain conserved sequence elements. This library was cloned into the AAV2 packaging vector with >100-fold sequence coverage and packaged into AAV2 virions. This library was subjected to the selective amplification step of VADER, either in the presence of the cognate EcTyrRS mutant that charge OMeY, or a non-cognate MbPylRS (FIG. 2a). Each selection was performed in duplicate and enrichment factor for each tRNA^{Tyr} mutant from these replicates were plotted against each other. Nine out of the ten tRNA^{Tyr} mutants that exhibit the highest enrichment in the presence of EcTyrRS (FIG. 2d) did not show strong enrichment when the non-cognate MbPylRS was used instead (FIG. 2e). These nine promising tRNA^{Tyr} sequences were synthesized and tested by co-transfecting them into HEK293T cells with OMeY-selective EcTyrRS and EGFP-39TAG in the presence and absence of 1 mM OMeY (FIG. 2f). Many of these mutants demonstrated significantly higher activity relative to wild-type-tRNA^{Tyr}, with the most active one showing an approximately three-fold improvement. Further evolution of this tRNA, as well as detailed characterization of the improved mutant isolated here are currently in progress. Success of this alternative VADER scheme, lacking the bioorthogonal capture step, further highlights the versatility of this platform for directed evolution of tRNAs and beyond.

Finally, improved mutants of E. coli tRNA_{CUA}^{Tyr}, were developed by subjecting an acceptor-stem library to a modified VADER scheme lacking the bioorthogonal capture step. These results suggest that it should be possible to extend the application of VADER to other suppressor tRNAs commonly used for genetic code expansion in mammalian cells. Additionally, it should be possible to adapt this platform for the directed evolution of other biological machinery for Uaa mutagenesis and beyond.

### Example 2: Cell Culture

HEK293T cells (ATCC, catalog number CRL-3216) were maintained at 37° C. and 5% CO2 in DMEM-high glucose (HyClone) supplemented with penicillin/streptomycin (HyClone, final concentration of 100 U/mL penicillin and 100 g/mL streptomycin) and 10% fetal bovine serum (Corning). All references to DMEM below refer to the complete medium described here.

### Example 3: Statistical Methods

For assessing EGFP reporter expression in HEK293T cells, the mean of at least three independent experiments was reported, and error bars represent s.d. For evaluating the enrichment factors at each selection step, three independent experiments were performed, and for each sample three independent measurements (FACS) were made and the average of these values were reported (error represents s.d.).

### Example 4: General Cloning

For all cloning, the E. coli TOP10 strain was used for transformation and plasmid propagation and bacteria were grown using LB for solid and liquid culture. All PCR reactions were carried out using Phusion Hot Start II DNA Polymerase (Thermo Scientific) according to the manufacturer's protocol. Restriction enzymes and T4 DNA ligase were from New England Biolabs (NEB). All DNA oligos were purchased from Integrated DNA Technologies (IDT). Sanger sequencing was performed by Eton Bioscience.

### Unnatural Amino Acids

Azido-lysine (AzK) was purchased from Iris Biotech GMBH (Germany). N^{ε}-acetyllysine (AcK) was purchased from Bachem. Diazirine-lysine (DiazK) was purchased from Sirius Fine Chemicals (Germany). Strained cyclooctyne-L-lysine (SCOK) was purchased from Sirius Fine Chemicals. O-Methyl-L-tyrosine (OMeY) was purchased from Thermo Scientific. Other suitable tyrosine or phenylalanine analogs (e.g., Fmoc-O-methyl-L-tyrosine; p-azido-L-phenylalanine (AzF) or Fmoc-beta-methyl-DL-phenylalanine) can be purchased from, for example, AnaSpec, Fremont, CA 94555.

### Plasmids

The AAV2 production plasmids pHelper and pAAV-RC2 were purchased from Cell Biolabs. pIDTSmart-RC2(T454TAG)-MbPylRS has been previously described. (Kelemen, R. E. et al. A Precise Chemical Strategy To Alter the Receptor Specificity of the Adeno-Associated Virus. Angewandte Chemie (International ed. in English) 55, 10645-10649, doi:10.1002/anie.201604067 (2016)).

pIDTSmart-PytR plasmids with anticodons for TAG, TGA, and TAA have been previously described. (Zheng, Y., Addy, P. S., Mukherjee, R. & Chatterjee, A. Defining the current scope and limitations of dual noncanonical amino acid mutagenesis in mammalian cells. Chem Sci 8, 7211-7217, doi:10.1039/c7sc02560b (2017)).

pAcBac1-EGFP has been previously described (Chatterjee, A., Xiao, H., Bollong, M., Ai, H. W. & Schultz, P. G. Efficient viral delivery system for unnatural amino acid mutagenesis in mammalian cells. Proceedings of the National Academy of Sciences of the United States of America 110, 11803-11808, doi:10.1073/pnas.1309584110 (2013)).

pAAV-ITR-PytR-GFP was generated by amplifying the U6-tRNA cassette from pIDTSmart-PytR (Zheng, Y., Lewis, T. L., Jr., Igo, P., Polleux, F. & Chatterjee, A. Virus-Enabled Optimization and Delivery of the Genetic Machinery for Efficient Unnatural Amino Acid Mutagenesis in Mammalian Cells and Tissues. ACS synthetic biology, doi:10.1021/acssynbio.6b00092 (2016) with primers tRNA-AAV-F and R and inserting into the MluI site of the AAV cargo plasmid pAAV-GFP (purchased from Cell Biolabs). A KpnI site was added before the U6 promoter using primer tRNA-Amp-F. pAAV-ITR-EcYtR-GFP was constructed in an analogous manner by amplifying the U6-tRNA cassette from pIDTSmart-YtR; pAAV-ITR-PytR-mCherry was generated by replacing the GFP reporter in pAAV-ITR-PytR-GFP; and pAAV-ITR-EcYtR-mCherry was generated by replacing the GFP reporter in pAAV-ITR-EcYtR-GFP.

The pAAV-ITR-GFP library cloning vector was generated by cutting pAAV-ITR-PytR-GFP at two NdeI sites flanking the tRNA and ligating the resulting vector back together. This vector retains the library cloning sites but lacks a tRNA which could cause background issues during library cloning.

pIDTSmart-1×PytR-evolved was generated by amplifying the best hit tRNA (Ac2.1 GGG/CCU) from selection using primers tRNA-Amp-F and R and cloning this insert into the pIDTSmart-PytR backbone using AvrII and NheI. Anticodons were mutated using site-directed mutagenesis.

pIDTSmart-MbPylRS, pIDTSmart-AcKRS3, pIDTSmart-HpRS, pIDTSmart-AbKRS, and pIDTSmart-OMeYRS were generated by cloning each synthetase into the pIDTSmart backbone at the AvrII and NheI sites.

### Example 5: Library Generation

Pyrrolysyl tRNA libraries were generated by site-saturation mutagenesis using pAAV-PytR-GFP as a template. For acceptor stem libraries, the 5′ and 3′ pieces of the tRNA were first amplified using primer pairs tRNA-Amp-F+AccLib-Short-R and AccLib-Short-F+tRNA-Amp-R respectively. Randomized bases were then added by reamplifying each fragment, replacing AccLib-Short-F and R with AccLib1 or AccLib2-NNN-F and R. Finally, the fragments were joined by overlap extension PCR and then amplified using tRNA-Amp-F and R, digested with KpnI and NcoI, and ligated into the pAAV-ITR-GFP library vector which had been digested with the same enzymes. Each ligation used ~1 μg each of vector and insert. T stem library generation was similar, but for each library, only one primer containing randomized nucleotides was used. The Tyrosyl tRNA library was generated using the same method with pAAV-EcYtR-GFP as a template and YtR-AccLib-Short-F, YtR-AccLib-Short-R, YtR-AccLib1-F, and YtR-AccLib1-R in the stead of the corresponding tRNA¹¹ primers.

Ligations were concentrated by ethanol precipitation with yeast tRNA (Ambion) and transformed into electrocompetent TOP10 E. coli. >4×10⁵ transformants were plated (>100-fold library coverage). These colonies were pooled and their DNA was miniprepped for packaging into AAV.

### Example 6: Packaging and Titration of Mock and Library tRNAs into AAV (Wild-Type Capsid)

To package various cargo into AAV2, 8 million HEK293T cells were seeded in a 10 cm tissue culture dish. The following day, the cells were transfected with 8 g each of the appropriate cargo plasmid (pAAV-ITR-tRNA-fluorescent protein), pHelper, and pAAV-RC2 using polyethylenimine (PEI) (Sigma). Media was exchanged for fresh DMEM 24 hours after transfection. 72 hours after transfection, the cells were resuspended, pelleted, and lysed by freeze/thawing as previously described. (Kelemen, R. E. et al. A Precise Chemical Strategy To Alter the Receptor Specificity of the Adeno-Associated Virus. Angewandte Chemie (International ed. in English) 55, 10645-10649, doi:10.1002/anie.201604067 (2016); Kelemen, R. E., Erickson, S. B. & Chatterjee, A. Production and Chemoselective Modification of Adeno-Associated Virus Site-Specifically Incorporating an Unnatural Amino Acid Residue into Its Capsid. Methods in molecular biology (Clifton, N.J.) 1728, 313-326, doi:10.1007/978-1-4939-7574-7_20 (2018)).

Virus was concentrated and semi-purified by PEG precipitation, as described in Kelemen above, resuspended in 1 mL DMEM with FBS, and flash frozen.

### Example 7: FACS Analysis to Assess Infective Titer of AAV2 Samples

Infective titers for virus preparations were determined using flow cytometry. 0.7 million HEK293T cells were seeded in each well of a 12-well plate. Next day, confluent cells (~1 million cells per well) were infected with a dilution of the AAV2 to be titered (to achieve low MOI conditions). 5 mM sodium butyrate (Sigma) was added to boost infectivity and transgene expression. Two days later, the cells were trypsinized, washed with PBS, and analyzed by flow cytometry to count the fluorescent population.

### Example 8: Transfection to Determine Overexpression of AAV Genes with Rep and AdHelper

0.7 million HEK293T cells per well were seeded in a 12-well plate and infected the next day with AAV2 carrying a tRNA^{Pyl}-mCherry cargo at an MOI of 1. Cells were transfected four hours after infection with 0.6 g pAAV-RC2 and 1.4 g pHelper. PEI-only negative control wells received the equivalent amount of PEI to the transfected wells, but no plasmids. Virus-only wells were not transfected at all. Three days after infection and transfection, cells were lysed with CelLytic M buffer (Sigma) and mCherry fluorescence was measured on a Molecular Devices SpectraMax M5 microplate reader. The background from an uninfected well was subtracted.

### Example 9: Positive Selection

8 million HEK293T cells each were seeded in three 10 cm tissue culture dishes. The next day, the cells were infected with virus containing a tRNA library at an apparent MOI of 5 (the actual MOI is substantially reduced in the presence of PEI, the transfection reagent). Four hours after infection, the cells were transfected with 22 g of pHelper and 10 g of pIDTSmart-RC2(T454TAG)-RS per dish using PEI. 1 mM Uaa was also added at this point. For the tRNA^{Pyl} library, pIDTSmart-RC2(T454TAG)-PylRS was added in the presence of AzK. For the tRNATyr library, pIDTSmart-RC2(T454TAG)-OMeYRS was added in the presence of OMeY for the +OMeYRS selections and pIDTSmart-RC2(T454TAG)-PylRS was added in the presence of OMeY for the −OMeYRS selections. One day after transfection, the culture media was exchanged with fresh DMEM containing 1 mM Uaa. Cells were harvested three days after transfection and lysed as for virus isolation. The culture media was saved and recombined with clarified lysate, and this mixture was treated with 500 U universal nuclease (Thermo Scientific) for 30 minutes. Virus was recovered by PEG precipitation using 11% polyethylene glycol (Fisher) as previously described^{26,28} and resuspended in 3 mL PBS.

The small-scale mock positive selections were carried out in 12-well plates. 0.7 million cells per well were seeded and infected the next day with AAV carrying a tRNA^{Pyl}-mCherry cargo. Four hours later the cells were transfected as described for the above selections, but with the transfection mix and AzK scaled down by a factor of 15. For PEI only wells, cells received a comparable amount of transfection reagent but no plasmid. Media was changed the day after transfection for fresh DMEM containing 1 mM AzK. Virus was harvested three days post-transfection and PEG-precipitated as described for the selections above. Confluent cells in a 12-well plate were infected with the entire output of one mock selection well and analyzed by flow cytometry.

### Example 10: Negative Selection—Streptavidin Pulldown

The virus from positive selection (3 mL) was labeled with photocleavable DBCO-sulfo-biotin (Jena Biosciences) at a concentration of 5 μM for one hour in the dark with mixing. Immediately after labeling, excess DBCO-biotin was quenched with AzK (1 mM final concentration) and the reactions were dialyzed overnight using Slide-A-Lyzer 100 kDa MWCO devices (Thermo Scientific) against 1 L PBS at 4° C. The dialyzed virus mixtures were split into three 2 mL tubes and each rotated overnight with 400 μL streptavidin agarose resin (Thermo Scientific) at 4° C. The next day, each tube of beads was washed eight times with 1 mL PBS containing additional NaCl (final concentration 300 mM) with mixing between washes. Finally, the washed beads were resuspended in 8 mL PBS (300 mM NaCl) and the virus was eluted from the resin via four 30-second irradiations using a 365 nm UV diode array (Larson Electronics), with mixing between irradiations.

### Example 11: Viral DNA Recovery, Amplification, and Cloning

The eluted virus was concentrated from 3 mL to 300 μL using Amicon Ultra-4 100 kDa MWCO centrifugal concentrators (Millipore). This mixture was heated to 100° C. for 10 minutes in order to denature the viral capsid proteins and expose the DNA. Viral DNA was then cleaned up and concentrated by ethanol precipitation using yeast tRNA (Ambion) and resuspended in a final volume of 50 μL.

20 μL of this mixture was added to a 200 μL PCR reaction and amplified with tRNA-Amp-F and R primers. The resulting DNA was digested with KpnI and NcoI and cloned into the library cloning vector using the same protocol as for original library generation.

### Example 12: Mock Selections Using Pyl-mCherry and Tyr-GFP

The mock selections shown in FIG. 1 followed the same protocol as above, except that the starting library virus was a 1:10,000 mixture of virus made from pAAV-ITR-PytR-mCherry to virus made from pAAV-ITR-EcYtR-GFP. Mock selection results were analyzed by flow cytometry as described for virus titering, but here, cells in a 12-well plate were infected with 200 μL of the virus pool after either positive or negative selection. Red and green fluorescent cells were counted to determine the virus ratio.

### Example 13: Hit Sequencing and Characterization

For each tRNA^{Pyl} library, 30-50 colonies were picked from the transformation plates generated above and sent for Sanger sequencing (Eton Bioscience). All sequences in which all randomized bases were paired were treated as potential hits, and these tRNAs were subcloned into pAAV-ITR-PytR-mCherry for further analysis.

Initial hit analysis was conducted by transfecting HEK293T cells in 24-well plates with 0.5 g each of a potential hit pAAV-ITR-PytR-mCherry plasmid, pIDTSmart-MbPylRS, and pAcBac1-GFP(39TAG) in the presence and absence of 1 mM AzK. Two days after transfection, cells were lysed with CelLytic M buffer (Sigma) and EGFP and mCherry fluorescence were measured on a Molecular Devices SpectraMax M5 microplate reader. Values for an untransfected well were subtracted, and EGFP fluorescence was normalized to mCherry fluorescence for each well.

The best hit, A2.1 (GGG/CCU), was selected for further analysis with other stop codons and synthetases. HEK293T cells in a 12-well plate were transfected with 0.375 g pIDTSmart-PytR containing either the wild-type or evolved tRNA, 0.375 g pIDTSmart-aaRS containing the appropriate synthetase, and 0.75 g pAcBac1-EGFP containing one or two of the appropriate stop codon. A wild-type EGFP control well used pIDTSmart-PytR(TAG, wild-type), pIDTSmart-MbPylRS, and pAcBac1-EGFP(wild-type) in the same ratios. Two days after transfection, cells were lysed and EGFP fluorescence was measured by microplate reader. Values from an untransfected well were subtracted.

### Example 14: Mass Spectrometry

Incorporation of the correct Uaas by the evolved tRNA was confirmed by LC-ESI-MS. EGFP-39AzK-6×His was generated by transfecting HEK293T cells in two 10 cm tissue culture dishes with 5 g pIDTSmart-PytR-evolved, 5 g pIDTSmart-MbPylRS, and 10 g pAcBac1-GFP(Y39TAG) with 1 mM AzK. EGFP-39AcK-6×His was generated by transfecting HEK293T cells in two 10 cm tissue culture dishes with 5 g pIDTSmart-PytR-evolved, 5 g pIDTSmart-AcKRS3, and 10 g pAcBac1-GFP(Y39TAG) with 5 mM AcK. Cells were lysed two days after transfection using CelLytic M (Sigma), Halt protease inhibitor cocktail (Thermo Scientific), and universal nuclease (Thermo Scientific) according to the manufacturers' instructions. All proteins were isolated from clarified lysate on Ni-NTA columns using HisPur resin (Fisher) according to the manufacturer's instructions, but using 60 μL of resin and wash buffers containing 30 mM and then 40 mM imidazole. Proteins were analyzed by LC-ESI-MS using an Agilent 1260 Infinity ESI-TOF.

### Example 15: Illumina Sample Preparation and Sequencing

To enable sequencing by the Illumina MiSeq System, DNA samples to be sequenced had Illumina adapter sequences attached via two rounds of PCR. The two consecutive reactions result in construction of the Illumina adapters provided by the TruSeq DNA HT Sample Prep Kit (Illumina). The forward adapter is AATGATACGGCGACCACCGAGATCTACAC[i5]ACACTCTTTCCCTACACGACGCTCTT CCGATCT (SEQ ID NO:32), wherein [i5] is an eight-nucleotide barcode sequence, and the reverse adapter is GATCGGAAGAGCACACGTCTGAACTCCAGTCAC[i7]ATCTCGTATGCCGTCTTCTGCT TG (SEQ ID NO:33), wherein [i7] is an eight-nucleotide barcode sequence. The first set of primers, Illumina-PytR-F and Illumina-PytR-R, consist of half of the TruSeq adapters, beginning immediately after the i5 or i7 barcode, followed by primer-binding sites to anneal to the sequences surrounding the tRNA library (for the forward primer: TTATATATCTTGTGGAAAGGACGAAAC (SEQ ID NO:34); for the reverse primer: GCTAGCGGATCGACGAGAGC (SEQ ID NO:35)). The second set of primers, a series of Illumina-i5-F and Illumina-i7-R variants containing different barcodes, consists of the 5′ half of the TruSeq adapters, followed by an i5 or i7 barcode, followed by primer-binding sites to anneal to the first PCR (for the forward primer: ACACTCTTTCCCTACACGACGC (SEQ ID NO:36); for the reverse primer: GTGACTGGAGTTCAGACGTGTGCTC (SEQ ID NO:37)).

Barcodes used in Illumina sequencing to enable sample multiplexing: Barcode Sequence i5-D501 TATAGCCT i5-D502 ATAGAGGC i5-D503 CCTATCCT i5-D504 GGCTCTGA i7-D701 CGAGTAAT i7-D702 TCTCCGGA i7-D703 AATGAGCG i7-D704 GGAATCTC.

For the first PCR, samples were prepared using the primers Illumina-PytR-F and Illumina-PytR-R and PrimeSTAR Max DNA Polymerase (Takara Bio) per manufacturer's instructions. PCR samples were purified by agarose gel extraction. A second round of PCR using Illumina-i5-F and Illumina-i7-R primer variants and PrimeSTAR Max DNA Polymerase was performed to attach the region of the adapter sequences that includes the barcodes. Unique combinations of i5 and i7 barcode sequences were applied to each sample to enable multiplex sequencing. Although both forward and reverse reads were possible, only forward reads were used in data analysis for this paper.

Samples were prepared for sequencing using the 300-cycle MiSeq Reagent Kit v2 (Illumina) per manufacturer's instructions. Sequencing was executed on an Illumina MiSeq System, with 10% Illumina PhiX Control added.

### Example 16: Illumina High-Throughput-Sequencing Data Processing

Processing of sequencing data generated by the MiSeq System was performed using bespoke Python scripts. Scripts are available via GitHub (Kelemen, R. (2018) VADER processing pipeline (Version 1.0) [github.com/chatterjeelab2022/VADER]).

Reads were first filtered by data quality. This “Q-score filter” checked the Phred base call quality scores associated with each read in the FASTQ output files generated by the MiSeq System. Reads below a specified threshold were discarded. For this study, reads containing any bases with Q-scores lower than 14, corresponding to an error rate of 4%, were discarded. Reads were then filtered by alignment with the expected tRNA sequence. This “mismatch filter” compared each read to the expected tRNA sequence within the fixed regions of the tRNA, skipping over the randomized regions within the tRNA associated with each library. Finally, a minimum abundant library count could be specified to reduce very rare sequences that may be the result of an error. However, for these samples, the minimum abundant library count was set to 1; no library members were discarded for being too low in abundance.

For sequences that pass both the Q-score filter and the mismatch filter, the sequence regions randomized in each library were extracted and collected in a comma-separated file. For each sequence in a given sample, the “fraction of total” value was calculated by dividing the counts of a given sequence by the total counts of all sequences in that sample. The number of base pairs found within the randomized library region of each sequence was also calculated, because successful base-pairing with stem regions is important for tRNA activity.

For each library, three samples were sequenced: the input library and the output from two replicates of the selection. The counts detected for each individual sequence in each sample were tallied. For each selection, the “fold enrichment” of a given library member was determined by calculating the ratio of counts in the selection output to counts in the selection input. The “enrichment factor” of a given library member is the fold enrichment normalized to the most enriched hit, and was determined by dividing the fold enrichment of a given hit by the fold enrichment of the most enriched hit in that sample. Finally, for each library member, the average of the two enrichment factors obtained was determined, and library members were sorted by average enrichment factor.

### Example 17: RNA Isolation

To generate RNA for Northern blots, 7.5 million HEK293T cells were seeded in a 10 cm tissue culture dish. The following day, cells were transfected at 75% confluency with 24 μg of tRNA variant-containing pAAV-ITR-PytR-mCherry plasmid using polyethylenimine (PEI) (Sigma). Media was exchanged for fresh DMEM 24 h after transfection, and cells were harvested 48 h post-transfection. The mCherry reporter expression was used to confirm comparable transfection efficiency between experiments. RNA isolation was performed with TRIzol Reagent (Thermo Fisher) following manufacturer's instructions. RNA concentration was determined via Nanodrop Spectrophotometer and integrity of the RNA was assessed by A260/A280 value, as well as the presence of distinct, intact 28S and 18S ribosomal RNA bands on 1% agarose gel.

### Example 18: Northern Blot Probe Preparation

Oligonucleotides were 3′-end labeled with DIG using the DIG Oligonucleotide 3′-End Labeling Kit, 2nd Generation (Roche), following manufacturer's instructions. Final concentration of labelled probe was determined per manufacturer's instructions. The oligonucleotide used was PyltR-Tstem2-NB-R, which binds to the T-stem of the tRNA in order to be compatible with all A-stem variations. For detection of the 5.8S RNA positive control to assess overall RNA concentration, the oligonucleotide 5.8S-NB-R was used.

### Example 19: Northern Blotting

A sensitive non-isotopic northern blot was performed using a previously established method of digoxigenin (DIG)-labeled oligonucleotide probes and 1-ethyl-3-(3-dimethylaminopropyl) carbodiimide for RNA-membrane cross-linking, with modification.⁵¹ Denaturing gels were 8 M Urea, 6.5% acrylamide in Tris/Borate/EDTA buffer (TBE), and made in mini-gel format (10.1×7.3 cm² with 1.5 mm spacers). Gel wells were thoroughly rinsed with TBE after gel was set and again after pre-running gel at 250 V for 60 minutes in TBE running buffer. 5 g of total RNA was combined with 2.5 μL Invitrogen™ Gel Loading Buffer II 2x (Denaturing PAGE, Fisher) to prepare a final sample volume of 5 μL per lane. Both RNA samples and RNA ladder (Low Range ssRNA Ladder, New England Biolabs) were denatured at 95° C. for 5 min, chilled on ice for 2 min, and then loaded onto gel. Gel was run at 4° C. for 70 minutes and then soaked in 0.05% ethidium bromide solution in RNAse free water while shaking for 10 minutes. The gel was imaged with the ChemiDoc-IT Imaging System and then soaked for 10 minutes in TBE running buffer.

A cassette sandwich was made of three sheets of 3MM Whatman paper soaked in TBE, nylon membrane, gel, and an additional three sheets of Whatman paper. Transfer of RNA from gel to membrane was done at 10 V for 60 minutes at 4° C. with the Trans-Blot SD Semi-Dry Transfer Cell (Bio-Rad).

EDC cross-linking solution was prepared as previously described.⁵¹ Whatman paper was saturated in EDC cross-linking solution and placed on top of Saran wrap. Membrane was placed on top of Whatman paper and allowed to incubate at 60° C. for 1 hour to facilitate RNA-membrane cross-linking. Residual cross-linking solution was then removed by thoroughly rinsing the membrane with distilled water.

DIG blocking, DIG washing, DIG detection buffer were prepared per manufacturer's instructions from DIG Wash and Block Buffer Set (Millipore Sigma). CSPD detection buffer was made with 50 μL of CSPD™ Substrate (0.25 mM Ready-To-Use, ThermoFisher) and 5 mL of detection buffer. DIG antibody solution was prepared by mixing DIG antibody (Anti-Digoxigenin-AP, Fab fragments) and blocking buffer at a ratio of 1:15,000. After cross-linking, membrane was incubated in 50 mL Falcon tube at 42° C. for 30 minutes in hybridization oven with 5 mL of Invitrogen™ ULTRAhyb™ Ultrasensitive Hybridization Buffer (Fisher), preheated to 68° C. The DIG-labelled probes PyltR-Tstem2-NB-R-DIG and 5.8S-NB-R-DIG were diluted to 25 nM and 2.5 nM, respectively, and denatured at 95° C. for 5 minutes. 5 μL of each probe was added to liquid in pre-hybridized Falcon tube and hybridized overnight at 42° C. in hybridization oven at slow rotation speed. The following day, membrane was washed through incubation twice with 5 mL Low Stringent Buffer (2×SSC with 0.1% SDS) at 42° C. for 5 minutes, twice with 5 mL High Stringent Buffer (0.1×SSC with 0.1% SDS) at 42° C. for 15 minutes, and then once with 10 mL Washing Buffer (1×SSC) at 42° C. for 10 minutes in hybridization oven. Membrane was incubated in 10 mL DIG blocking buffer at room temperature for 3 hours in hybridization oven. Membrane was then incubated with 10 mL of DIG antibody solution at room temperature for 30 minutes in hybridization oven. Membrane was then washed four times with 10 mL of DIG washing buffer for 15 minutes in hybridization oven and incubated with 5 mL of DIG detection buffer for 5 minutes at room temperature in hybridization oven. Membrane was then removed from tube with clean forceps, placed on plastic wrap, and incubated for 5 minutes with 5 mL of CSPD detection buffer. Membrane was then placed in a heat-sealable plastic bag, sealed, and incubated at 37° C. for 15 minutes in the dark. The membrane was then imaged for chemiluminescence for 10 minutes with the ChemiDoc-IT Imaging System.

### Example 20: Production of Baculovirus Vectors and their Use for Testing tRNA^{Pyl} Activity with Copy Number Control

VSVG-pseudotyped baculovirus vectors were generated and titered as previously described in Chatterjee, A., Xiao, H., Bollong, M., Ai, H. W. & Schultz, P. G. Efficient viral delivery system for unnatural amino acid mutagenesis in mammalian cells. Proceedings of the National Academy of Sciences of the United States of America 110, 11803-11808, doi:10.1073/pnas.1309584110 (2013). See also: Kim, S. W. et al. A sensitive non-radioactive northern blot method to detect small RNAs. Nucleic acids research 38, e98, doi:10.1093/nar/gkp1235 (2010).

### Data Availability:

The NGS data sets for the tRNA^{Pyl} and tRNA^{Tyr} experiments are available via Zenodo at accession codes doi:10.5281/zenodo.7186997 and doi:10.5281/zenodo.7187354, respectively. Zenodo is an open data repository maintained by (CERN). A digital object identifier (doi) is automatically assigned to all Zenodo files.

Code availability: All scripts used in this manuscript are available via GitHub. (Kelemen, R. (2018) VADER processing pipeline (Version 1.0) [github.com/chatterjeelab2022/VADER]).

While this invention has been particularly shown and described with references to preferred embodiments thereof, it will be understood by those skilled in the art that various changes in form and details may be made therein without departing from the scope of the invention encompassed by the appended claims.

1. A composition comprising a variant bacterial tyrosyl-suppressor tRNA (tyrosyl-tRNA), wherein the variant tyrosyl-tRNA has increased activity to incorporate a tyrosine or phenylalanine amino acid analog into a protein expressed in mammalian cells relative to its wild-type counterpart tRNA.

2. A viral vector comprising a variant bacterial tyrosyl-suppressor tRNA, wherein the variant tRNA has increased activity to incorporate a tyrosine or phenylalanine amino acid analog into a protein expressed in mammalian cells relative to its wild-type counterpart tRNA.

3. The composition of claim 1, wherein the activity of the variant tRNA is increased over the wild type tRNA by about 2.5 to 20 fold.

4. The composition of claim 1; wherein the variant bacterial tRNA is derived from an E. coli tyrosyl-tRNA (tRNA^{Tyr}) comprising SEQ ID NO:1.

5. The composition or viral vector of claim 4, wherein the variant tRNA is a tyrosyl-tRNA (tRNA^{Tyr}) comprising a sequence selected from the group consisting of: SEQ ID NOS: 3 through 31, or a nucleic acid sequence with at least about 90% sequence identity to any of the full-length sequences of SEQ ID NOS: 3-31.

6. The composition or viral vector of claim 1, wherein the tyrosine amino acid analog is selected from the group consisting of tyrosine analog structures 1-11.

7. A cell comprising the viral vector of claim 2, wherein the cell is a mammalian cell.

8. The mammalian cell of claim 7, wherein the viral vector is adeno-associated virus and the essential viral protein is a TAG-mutant of Cap (SEQ ID NO:2).

9. The mammalian cell of claim 7, wherein the cell further comprises plasmids encoding:
a) a protein essential for viral replication, wherein a nonsense codon is inserted into a protein sequence rendering viral replication dependent on the activity of the variant suppressor tRNA;
b) a cognate Uaa RNA Synthetase (UaaRS); and
c) genetic components required for viral replication.

10. The mammalian cell of claim 9, wherein the cognate aaRS is E. coli^{tyr} RS.

11. A method of virus-assisted directed evolution of orthogonal tyrosyl-suppressor tRNA variants of interest with increased biological activity relative to the wild-type suppressor tRNA, wherein the replication of the virus in mammalian cells requires expression of a nonsense mutant of an essential viral protein which is dependent on the activity of the suppressor tRNA variant of interest, the method comprising the steps of:
a) encoding a library of suppressor tRNA variants in a virus genome;
b) infecting a population of mammalian host cells with the virus vectors at low multiplicity of infection (MOI);
c) subsequently introducing into the population of mammalian host cells additional plasmids/genetic elements, wherein the plasmids/genetic elements comprise:
i) a protein essential for viral replication, wherein a nonsense codon is inserted into the protein sequence rendering viral replication dependent on the activity of the variant suppressor tRNA;
ii) a cognate Uaa RNA Synthetase (UaaRS); and
iii) genetic components required for viral replication;
d) substantially simultaneously adding a suitable unnatural amino acid to the culture media;
e) maintaining the infected/transfected cells in the media under conditions suitable for replication of the virus;
f) harvesting the cells and isolating virus progeny;
g) optionally labeling the virus isolated in step f) with a purification handle attached through a photocleavable linker and recovering labeled virus through enrichment followed by release using irradiation at a suitable wavelength;
h) lysing the recovered virus and amplifying tyrosyl-tRNA variants contained in the lysate whereby orthogonal suppressor tyrosyl-tRNA variants with increased biological activity are recovered.

12. The method of claim 11, wherein the essential viral protein is the capsid protein (CAP) (SEQ ID NO:2) of the adeno associated virus and is mutated to include a stop codon at position 454 of the protein.

13. The method of claim 11, wherein no more than a single virion infects the host mammalian cell.

14. A method of producing a protein in a mammalian cell with one, or more, tyrosine or phenylalanine amino acid analogs at specified positions in the protein, the method comprising,
a. culturing the mammalian cell in a culture medium under conditions suitable for growth, wherein the cell comprises a nucleic acid that encodes a protein with one, or more, selector codons,
wherein the cell further comprises a variant E. coli-derived Tyr-tRNA with increased biological activity that recognizes the selector codon, and its cognate amino acyl-RNA synthetase, and
b. contacting the cell culture medium with one, or more, tyrosine or phenylalanine amino acid analogs under conditions suitable for incorporation of the one, or more, tyrosine or phenylalanine analogs into the protein in response to the selector codon,
thereby producing the protein with one, or more tyrosine or phenylalanine analogs incorporated therein.

15. The method of claim 14, wherein the variant E. coli-derived Tyr-tRNA comprises a nucleic acid sequence selected from the group consisting of: SEQ ID NOS: 3-31, or a nucleic acid sequence with at least about 90% sequence identity to any of the full-length sequences of SEQ ID NOS: 3-31.

16. A method of site-specifically incorporating one, or more, tyrosine or phenylalanine amino acid analogs into a protein or peptide produced in a mammalian cell, the method comprising,
a. culturing the mammalian cell in a culture medium under conditions suitable for growth, wherein the cell comprises a nucleic acid that encodes a protein or peptide of interest with one, or more, amber, ochre or opal selector codons at specific sites in the protein or peptide,
wherein the cell further comprises a variant E. coli-derived tRNA^{Tyr} with increased biological activity that recognizes the selector codon, and further comprises an E. coli Tyr-RNA Synthetase;
b. contacting the cell culture medium with one, or more, tyrosine or phenylalanine analog residues under conditions suitable for incorporation of the one, or more, tyrosine analog residues into the protein or peptide at the sites of the selector codon(s),
thereby producing the protein or peptide of interest in a mammalian cell with one, or more site-specifically incorporated tyrosine or phenylalanine analog residues.

17. The method of claim 16, wherein the variant E. coli-derived Tyr-tRNA comprises a nucleic acid sequence selected from the group consisting of: SEQ ID NOS: 3-31 or a nucleic acid sequence with at least about 90% sequence identity to any of the full-length sequences of SEQ ID NOS: 3-31.

18. A kit for producing a protein or peptide of interest in a mammalian cell, wherein the protein or peptide comprises one, or more tyrosine analogs, the kit comprising:
a. a container containing a polynucleotide sequence encoding variant E. coli derived tRNA^{Tyr} with increased biological activity that recognizes a selector codon in a nucleic acid of interest in a cell; and
b. a container containing a polynucleotide sequence encoding E. coli Tyr-tRNA synthetase.

19. The kit of claim 18, wherein the variant E. coli-derived Tyr-tRNA comprises a nucleic acid sequence selected from the group consisting of: SEQ ID NOS: 3-31 or a nucleic acid sequence with at least about 90% sequence identity to any of the full-length sequences of SEQ ID NOS: 3-31.

20. The kit of claim 18, wherein the kit further comprises a tyrosine or phenylalanine amino acid analog.

21. The kit of claim 18, wherein the kit further comprises instructions for producing the protein or peptide of interest.

22. A genetically-engineered mammalian cell with a stably integrated variant suppressor tRNA^{Tyr} for Uaa incorporation.

23. The genetically-engineered mammalian cell of claim 22 that comprises less than 250, 200, 150, 100, 75, 50 copies of a gene encoding a variant suppressor tRNA^{Tyr} capable of incorporating an unnatural amino acid into a protein of interest.

24. The genetically-engineered mammalian cell of claim 22, wherein the cell comprises 25-250, 25-200, 25-150, 25-100, 25-75, 25-50, 50-250, 50-200, 50-150, 50-100, 50-75, 75-250, 75-200, 75-250, 75-100, 100-250, 100-200, or 100-150 copies of the gene encoding the suppressor tRNA.

25. The genetically-engineered mammalian cell of claim 22, wherein the variant suppressor tRNA^{tyr} comprises a nucleic acid sequence selected from the group consisting of: SEQ ID NOS: 3-31, or a sequence with at least about 90% sequence identity to any of the full-length sequences of SEQ ID NOS: 3-31.


SEQUENCE LISTING (37 sequences)

SEQ ID NO: 1 - RNA, 82 nt, Escherichia coli
  misc_feature 1..82: note = Description of sequence: 0
  source 1..82: mol_type = tRNA
ggtggggttcccgagcggccaaagggagcagactctaaatctgccgtcacagacttcgaaggttcgaatccttcccccacca

SEQ ID NO: 2 - protein, 735 aa, synthetic construct [sequence omitted]

SEQ ID NO: 3 - RNA, 85 nt, synthetic construct
  misc_feature 1..85: note = Description of sequence: EcTyr mutant: HTS-8
  source 1..85: mol_type = other RNA
ggggcggttcccgagcggccaaagggagcagactctaaatctgccgtcacagacttcgaaggttcgaatccttcccgctccacca

SEQ ID NO: 4 - RNA, 85 nt, synthetic construct
  misc_feature 1..85: note = Description of sequence: EcTyr mutant: HTS-3
  source 1..85: mol_type = other RNA
ggggaggttcccgagcggccaaagggagcagactctaaatctgccgtcacagacttcgaaggttcgaatccttccctctccacca

SEQ ID NO: 5 - RNA, 85 nt, synthetic construct
  misc_feature 1..85: note = Description of sequence: EcTyr mutant: HTS-10
  source 1..85: mol_type = other RNA
gggggcgttcccgagcggccaaagggagcagactctaaatctgccgtcacagacttcgaaggttcgaatccttccgcctccacca

SEQ ID NO: 6 - RNA, 85 nt, synthetic construct
  misc_feature 1..85: note = Description of sequence: EcTyr mutant: NGS-6
  source 1..85: mol_type = other RNA
gggacggttcccgagcggccaaagggagcagactctaaatctgccgtcacagacttcgaaggttcgaatccttcccgtcccacca

SEQ ID NO: 7 - RNA, 85 nt, synthetic construct
  misc_feature 1..85: note = Description of sequence: EcTyr mutant: HTS-5
  source 1..85: mol_type = other RNA
ggggcggttcccgagcggccaaagggagcagactctaaatctgccgtcacagacttcgaaggttcgaatccttcccgccccacca

SEQ ID NO: 8 - RNA, 85 nt, synthetic construct
  misc_feature 1..85: note = Description of sequence: EcTyr mutant: HTS-11
  source 1..85: mol_type = other RNA
gggggagttcccgagcggccaaagggagcagactctaaatctgccgtcacagacttcgaaggttcgaatccttcctcctccacca

SEQ ID NO: 9 - RNA, 85 nt, synthetic construct
  misc_feature 1..85: note = Description of sequence: EcTyr mutant: HTS-13
  source 1..85: mol_type = other RNA
gggggggttcccgagcggccaaagggagcagactctaaatctgccgtcacagacttcgaaggttcgaatccttcgggggccacca

SEQ ID NO: 10 - RNA, 85 nt, synthetic construct
  misc_feature 1..85: note = Description of sequence: EcTyr mutant: HTS-12
  source 1..85: mol_type = other RNA
gggggagttcccgagcggccaaagggagcagactctaaatctgccgtcacagacttcgaaggttcgaatccttcctcccccacca

SEQ ID NO: 11 - RNA, 85 nt, synthetic construct
  misc_feature 1..85: note = Description of sequence: EcTyr mutant: HTS-9
  source 1..85: mol_type = other RNA
gggggggttcccgagcggccaaagggagcagactctaaatctgccgtcacagacttcgaaggttcgaatccttccccctccacca

SEQ ID NO: 12 - RNA, 85 nt, synthetic construct
  misc_feature 1..85: note = Description of sequence: EcTyr mutant: HTS-7
  source 1..85: mol_type = other RNA
gggccggttcccgagcggccaaagggagcagactctaaatctgccgtcacagacttcgaaggttcgaatccttcccggtccacca

SEQ ID NO: 13 - RNA, 85 nt, synthetic construct
  misc_feature 1..85: note = Description of sequence: EcTyr mutant: NGS-3
  source 1..85: mol_type = other RNA
gggccggttcccgagcggccaaagggagcagactctaaatctgccgtcacagacttcgaaggttcgaatccttcccggcccacca

SEQ ID NO: 14 - RNA, 85 nt, synthetic construct
  misc_feature 1..85: note = Description of sequence: EcTyr mutant: NGS-9
  source 1..85: mol_type = other RNA
ggcgcggttcccgagcggccaaagggagcagactctaaatctgccgtcacagacttcgaaggttcgaatccttcccgcgccacca

SEQ ID NO: 15 - RNA, 85 nt, synthetic construct
  misc_feature 1..85: note = Description of sequence: EcTyr mutant: NGS-7
  source 1..85: mol_type = other RNA
ggagcggttcccgagcggccaaagggagcagactctaaatctgccgtcacagacttcgaaggttcgaatccttcccgctccacca

SEQ ID NO: 16 - RNA, 85 nt, synthetic construct
  misc_feature 1..85: note = Description of sequence: EcTyr mutant: NGS-8
  source 1..85: mol_type = other RNA
ggaccggttcccgagcggccaaagggagcagactctaaatctgccgtcacagacttcgaaggttcgaatccttcccggtccacca

SEQ ID NO: 17 - RNA, 85 nt, synthetic construct
  misc_feature 1..85: note = Description of sequence: EcTyr mutant: HTS-4
  source 1..85: mol_type = other RNA
ggcacggttcccgagcggccaaagggagcagactctaaatctgccgtcacagacttcgaaggttcgaatccttcccgtgccacca

SEQ ID NO: 18 - RNA, 85 nt, synthetic construct
  misc_feature 1..85: note = Description of sequence: EcTyr mutant: HTS-1
  source 1..85: mol_type = other RNA
gggcgggttcccgagcggccaaagggagcagactctaaatctgccgtcacagacttcgaaggttcgaatccttccctgcccacca

SEQ ID NO: 19 - RNA, 85 nt, synthetic construct
  misc_feature 1..85: note = Description of sequence: EcTyr mutant: NGS-4
  source 1..85: mol_type = other RNA
gggccagttcccgagcggccaaagggagcagactctaaatctgccgtcacagacttcgaaggttcgaatccttcctggcccacca

SEQ ID NO: 20 - RNA, 85 nt, synthetic construct
  misc_feature 1..85: note = Description of sequence: EcTyr mutant: NGS-5
  source 1..85: mol_type = other RNA
ggcgcggttcccgagcggccaaagggagcagactctaaatctgccgtcacagacttcgaaggttcgaatccttctcgcgccacca

SEQ ID NO: 21 - RNA, 85 nt, synthetic construct
  misc_feature 1..85: note = Description of sequence: EcTyr mutant: NGS-2
  source 1..85: mol_type = other RNA
ggacgggttcccgagcggccaaagggagcagactctaaatctgccgtcacagacttcgaaggttcgaatccttccccgtccacca

SEQ ID NO: 22 - RNA, 85 nt, synthetic construct
  misc_feature 1..85: note = Description of sequence: EcTyr mutant: HTS-6
  source 1..85: mol_type = other RNA
ggcgggtttcccgagcggccaaagggagcagactctaaatctgccgtcacagacttcgaaggttcgaatccttcacccgccacca

SEQ ID NO: 23 - RNA, 85 nt, synthetic construct
  misc_feature 1..85: note = Description of sequence: EcTyr mutant: NGS-1
  source 1..85: mol_type = other RNA
gggggcgttcccgagcggccaaagggagcagactctaaatctgccgtcacagacttcgaaggttcgaatccttccgcccccacca

SEQ ID NO: 24 - RNA, 85 nt, synthetic construct
  misc_feature 1..85: note = Description of sequence: EcTyr mutant
  source 1..85: mol_type = other RNA
ggggggtttcccgagcggccaaagggagcagactctaaatctgccgtcacagacttcgaaggttcgaatccttcacctcccacca

SEQ ID NO: 25 - DNA, 82 bp, synthetic construct
  misc_feature 1..82: note = Description of sequence: C19T
  source 1..82: mol_type = other DNA
ggtggggttcccgagcggtcaaagggagcagactctaaatctgccgtcacagacttcgaaggttcgaatccttcccccacca

SEQ ID NO: 26 - DNA, 82 bp, synthetic construct
  misc_feature 1..82: note = Description of sequence: A51T
  source 1..82: mol_type = other DNA
ggtggggttcccgagcggccaaagggagcagactctaaatctgccgtcactgacttcgaaggttcgaatccttcccccacca

SEQ ID NO: 27 - DNA, 82 bp, synthetic construct
  misc_feature 1..82: note = Description of sequence: 47-53GC
  source 1..82: mol_type = other DNA
ggtggggttcccgagcggccaaagggagcagactctaaatctgccggcacagccttcgaaggttcgaatccttcccccacca

SEQ ID NO: 28 - DNA, 82 bp, synthetic construct
  misc_feature 1..82: note = Description of sequence: 59-73CG
  source 1..82: mol_type = other DNA
ggtggggttcccgagcggccaaagggagcagactctaaatctgccgtcacagacttcgcaggttcgaatcctgcccccacca

SEQ ID NO: 29 - DNA, 82 bp, synthetic construct
  misc_feature 1..82: note = Description of sequence: 60-72GC
  source 1..82: mol_type = other DNA
ggtggggttcccgagcggccaaagggagcagactctaaatctgccgtcacagacttcgagggttcgaatccctcccccacca

SEQ ID NO: 30 - DNA, 82 bp, synthetic construct
  misc_feature 1..82: note = Description of sequence: WT-T2
  source 1..82: mol_type = other DNA
ggtggggttcccgagcggccaaagggagcagactctaaatctgccgtcacagacttcgcgggttcgaatcccgcccccacca

SEQ ID NO: 31 - DNA, 82 bp, synthetic construct
  misc_feature 1..82: note = Description of sequence: HTS10-T2
  source 1..82: mol_type = other DNA
gggacggttcccgagcggccaaagggagcagactctaaatctgccgtcacagacttcgcgggttcgaatcccgcccgtccca

SEQ ID NO: 32 - DNA, 70 bp, synthetic construct
  misc_feature 1..70: note = Description of sequence: TruSeq forward adapter
  source 1..70: mol_type = other DNA
  variation 30..37: note = n is any one of a, c, g and t
  misc_feature 30..37: note = location of [i5] insert
aatgatacggcgaccaccgagatctacacnnnnnnnnacactctttccctacacgacgctcttccgatct

SEQ ID NO: 33 - DNA, 64 bp, synthetic construct
  misc_feature 1..64: note = Description of sequence: TruSeq reverse adapter
  source 1..64: mol_type = other DNA
  variation 34..40: note = n is any one of a, c, g and t
  misc_feature 34..40: note = location of [i7] insert
gatcggaagagcacacgtctgaactccagtcacnnnnnnnatctcgtatgccgtcttctgcttg

SEQ ID NO: 34 - DNA, 27 bp, synthetic construct
  misc_feature 1..27: note = Description of sequence: forward primer
  source 1..27: mol_type = other DNA
ttatatatcttgtggaaaggacgaaac

SEQ ID NO: 35 - DNA, 20 bp, synthetic construct
  misc_feature 1..20: note = Description of sequence: reverse primer
  source 1..20: mol_type = other DNA
gctagcggatcgacgagagc

SEQ ID NO: 36 - DNA, 22 bp, synthetic construct
  misc_feature 1..22: note = Description of sequence: forward primer
  source 1..22: mol_type = other DNA
acactctttccctacacgacgc

SEQ ID NO: 37 - DNA, 25 bp, synthetic construct
  misc_feature 1..25: note = Description of sequence: reverse primer
  source 1..25: mol_type = other DNA
gtgactggagttcagacgtgtgctc